(19) United States
(12) Patent Application Publication
(10) Pub. No.: US 2014/0116434 A1
(43) Pub. Date: May 1, 2014

(54) Dry Powder Inhaler Compositions

(71) Applicant: GLAXO GROUP LIMITED, Brentford, Middlesex (GB)

(72) Inventor: Glenn Crater, North Mississauga (CA)

(73) Assignee: GLAXO GROUP LIMITED, Brentford, Middlesex (GB)

(21) Appl. No.: 14/124,270

(22) PCT Filed: Jun. 1, 2012
(86) PCT No.: PCT/EP2012/060442

§ 371 Date: Dec. 6, 2013

Related U.S. Application Data
(60) Provisional application No. 61/494,594, filed on Jun. 8, 2011.

(51) Int. Cl. A61K 31/439; A61K 31/58; A61K 31/137; A61M 15/00

(52) U.S. Cl. CPC A61K 31/439; A61M 15/0045; A61K 31/58; A61K 31/137
USPC 128/203.15; 514/305; 514/171; 546/133

(57) ABSTRACT

Dry powder inhalers comprising a muscarinic acetylcholine receptor antagonist and optionally a beta 2 agonist and/or a corticosteroid for use in the treatment of inflammatory or respiratory tract diseases, such as asthma or COPD.

### FIELD OF THE INVENTION

The present invention provides pharmaceutical products for use in the treatment of chronic obstructive pulmonary disease (COPD), asthma and related diseases.

More specifically, the present invention provides dry powder inhalers comprising a muscarinic receptor antagonist, particularly 4-[hydroxy(diphenyl)methyl]-1-{2-[(phenylmethyl)oxy]ethyl}-1-azoniabicyclo[2.2.2]octane bromide, present in an amount to deliver about 31 to 32 mcg/dose or about 15 to 16 mcg/dose of the free cation, and optionally a beta-2 adrenoreceptor agonist, particularly 4-{(1R)-2-[(6-{2-[(2,6-dichlorobenzyl)oxy]ethoxy}hexyl)amino]-1-hydroxyethyl}-2-(hydroxymethyl)phenol triphenylacetate, and/or optionally a corticosteroid, particularly 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate).

### BACKGROUND OF THE INVENTION

Selective β₂-adrenoreceptor agonists have been used in the prophylaxis and treatment of clinical conditions for which a bronchodilating agent has been indicated. Such conditions include diseases associated with airflow obstruction such as chronic obstructive pulmonary diseases (COPD) (e.g. chronic and wheezy bronchitis, emphysema), asthma, respiratory tract infection and upper respiratory tract disease (e.g. rhinitis, including seasonal and allergic rhinitis).

In particular, asthma and other related disorders are typically treated with beta-2 adrenergic receptor agonists (beta-2 agonists) as they provide a bronchodilator effect to the patient, resulting in relief from the symptoms of breathlessness. Within the beta-2 agonist class there are presently available short acting compounds for immediate relief, such as salbutamol, biltolterol, pirbuterol and terbutaline. There are also longer acting compounds commercially available, such as salmeterol and formoterol. Salmeterol is available by prescription for use twice daily in the treatment of asthma.

Over the last two decades, inhaled anticholinergic agents have become well established as well-tolerated and effective bronchodilators for the treatment of COPD. Treatment with anticholinergics significantly improves FEV₁, (forced expiratory volume in 1 second) resting and dynamic lung hyperinflation, symptoms and exercise capacity, and reduces COPD exacerbations. Currently, only a few inhaled anticholinergic bronchodilators are available: the short-acting ipratropium bromide (ipratropium; dosed four-times-a-day) and oxitropium bromide, and the long-acting tiotropium bromide (tiotropium; dosed once-daily).

WO 03/024439 describes compounds of the general formula:

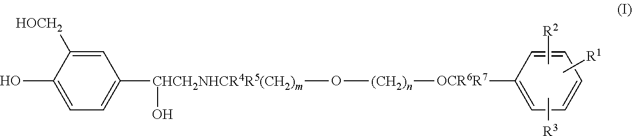

and salts, solvates, and physiologically functional derivatives thereof.

The compound 4-{(1R)-2-[(6-{2-[(2,6-dichlorobenzyl)oxy]ethoxy}hexyl)amino]-1-hydroxyethyl}-2-(hydroxymethyl)phenol is specifically described in WO03/024439, as are pharmaceutically acceptable salts thereof, in particular the acetate, triphenylacetate, α-phenylcinnamate, 1-naphthoate and (R)-mandelate salts.

WO2005/104745 describes compounds of the formulae:

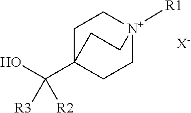

WO2005/104745 specifically describes the compound 4-[hydroxy(diphenyl)methyl]-1-{2-[(phenylmethyl)oxy]ethyl}-1-azoniabicyclo[2.2.2]octane bromide.

### SUMMARY OF THE INVENTION

The present invention is directed to a dry powder inhaler comprising a compound of formula (I):

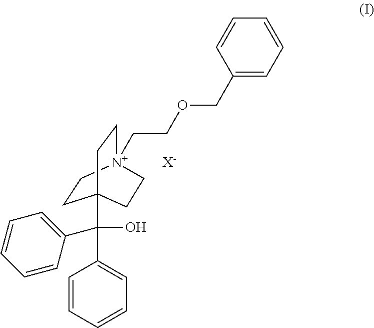

wherein X⁻ is a pharmaceutically acceptable anion and wherein the free cation of the compound of formula (I) is present in an amount of about 31 to 32mcg/dose or about 15 to 16 mcg/dose.

In a further embodiment, the present invention is directed to a dry powder inhaler as described above, which further comprises a compound of formula (II):

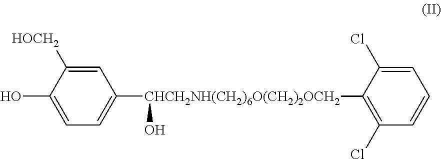

or a pharmaceutically acceptable salt thereof.

In one embodiment, the compound of formula (I) is 4-[hydroxy(diphenyl)methyl]-1-{2-[(phenylmethyl)oxy]ethyl}-1-azoniabicyclo[2.2.2]octane bromide.

In another embodiment, the pharmaceutically acceptable salt of the compound of formula (II) is the triphenylacetate salt.

The dry powder inhalers of the present invention may further comprise an inhaled corticosteroid, e.g. fluticasone propionate, mometasone furoate, budesonide, ciclesonide, or 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate).

In one embodiment, a dry powder inhaler of the present invention comprises 4-[hydroxy(diphenyl)methyl]-1-{2-[(phenylmethyl)oxy]ethyl}-1-azoniabicyclo[2. 2. 2]octane bromide present in an amount to deliver about 31 to 32 mcg/dose or about 15 to 16 mcg/dose of the free cation, 4-{(1R)-2-[(6-{2-[(2,6-dichlorobenzyl)oxy]ethoxy}hexyl)amino]-1-hydroxyethyl}-2-(hydroxymethyl)phenol triphenylacetate and 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate).

In a further embodiment, a dry powder inhaler of the present invention comprises 4-[hydroxy(diphenyl)methyl]-1-{2-[(phenylmethyl)oxy]ethyl}-1-azoniabicyclo[2.2.2]octane bromide present in an amount to deliver 31.25 mcg/dose or 15.625 mcg/dose of the free cation, 4-{(1R)-2-[(6-{2-[(2,6-dichlorobenzyl)oxy]ethoxy}hexyl)amino]-1-hydroxyethyl}-2-(hydroxymethyl)phenol triphenylacetate and 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate).

### DETAILED DESCRIPTION OF THE INVENTION

The present invention is directed to a dry powder inhaler comprising a compound of formula (I):

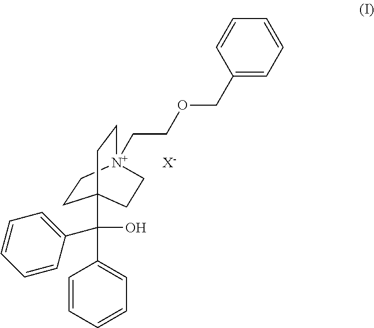

wherein X⁻ is a pharmaceutically acceptable anion and wherein the free cation of the compound of formula (I) is present in an amount of about 31 to 32 mcg/dose or about 15 to 16 mcg/dose.

In a further embodiment, the present invention is directed to a dry powder inhaler, wherein the free cation of the compound of formula (I) is present in an amount of about 31.25 mcg/dose or about 15.6 mcg/dose.

In yet a further embodiment, the present invention is directed to a dry powder inhaler, wherein the free cation of the compound of formula (I) is present in an amount of 31.25 mcg/dose or 15.6 mcg/dose.

In yet a further embodiment, the present invention is directed to a dry powder inhaler, wherein the free cation of the compound of formula (I) is present in an amount of 15.625 mcg/dose.

In a further embodiment, the present invention is directed to a dry powder inhaler as described directly above, which further comprises the compound of formula (II):

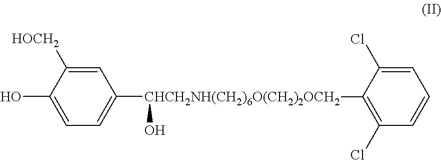

or a pharmaceutically acceptable salt thereof.

The pharmaceutically acceptable anion depicted by X⁻ may be selected from chloride, bromide, iodide, hydroxide, sulfate, nitrate, phosphate, acetate, trifluoroacetate, fumarate, citrate, tartrate, oxalate, succinate, mandelate, methanesulfonate or p-toluenesulfonate. In one embodiment the pharmaceutically acceptable anion X⁻ is bromide.

For purposes herein, the free cation of the compound of formula (I) is also referred to as 4-[hydroxy(diphenyl)methyl]-1-{2-[(phenylmethyl)oxy]ethyl}-1-azoniabicyclo[2.2.2]octane.

The compound of formula (II) is also referred to as 4-{(1R)-2-[(6-{2-[(2,6-dichlorobenzyl)oxy]ethoxy}hexyl)amino]-1-hydroxyethyl}-2-(hydroxymethyl)phenol.

In one embodiment, the compound of formula (I) is 4-[hydroxy(diphenyl)methyl]-1-{2-[(phenylmethyl)oxy]ethyl}-1-azoniabicyclo[2.2.2]octane bromide.

Pharmaceutically acceptable acid addition salts of the compound of formula (II) include those formed from hydrochloric, hydrobromic, sulphuric, citric, tartaric, phosphoric, lactic, pyruvic, acetic, trifluoroacetic, triphenylacetic, phenylacetic, substituted phenyl acetic eg. methoxy)phenyl acetic, sulphamic, sulphanilic, succinic, oxalic, fumaric, maleic, malic, glutamic, aspartic, oxaloacetic, methanesulphonic, ethanesulphonic, arylsulponic (for example p-toluenesulphonic, benzenesulphonic, naphthalenesulphonic or naphthalenedisulphonic), salicylic, glutaric, gluconic, tricarballylic, mandelic, cinnamic, substituted cinnamic (for example, methyl, methoxy, halo or phenyl substituted cinnamic, including 4-methyl and 4-methoxycinnamic acid and α-phenyl cinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids.

In one embodiment, the pharmaceutically acceptable salt of the compound of formula (II) is selected from the acetate, 1-naphthoate and (R)-mandelate salts.

In another embodiment, the pharmaceutically acceptable salt of the compound of formula (II) is the α-phenylcinnamate salt.

In another embodiment, the pharmaceutically acceptable salt of the compound of formula (II) is the triphenylacetate salt.

4-[hydroxy(diphenyl)methyl]-1-{2-[(phenylmethyl)oxy]ethyl}-1-azoniabicyclo[2.2.2]octane bromide has been the subject of studies in animal models, and in humans, and has been found to be a long acting high-affinity pan-active muscarinic receptor antagonist which has potential for once-daily administration.

4-{(1R)-2-[(6-{2-[(2,6-dichlorobenzyl)oxy]ethoxy}hexyl)amino]-1-hydroxyethyl}-2-(hydroxymethyl)phenol and its salts has been extensively tested in animal and human studies and has been found to demonstrate sustained bronchodilation over a 24 hour period in conjunction with a favourable safety profile and thus has the potential for once-daily administration.

The dry powder inhalers of the present invention, comprising the compound of formula (I) present in an amount to deliver about 31 to 32 mcg/dose or 15 to 16 mcg/dose of the free cation and optionally the compound of formula (II), or a pharmaceutically acceptable salt thereof, and/or optionally a corticosteroid, may have use in the treatment of inflammatory or respiratory tract diseases such as chronic obstructive pulmonary disease, chronic bronchitis, asthma, chronic respiratory obstruction, pulmonary fibrosis, pulmonary emphysema, allergic rhinitis, small airways disease, bronchiectasis and cystic fibrosis.

COPD is a chronic disease characterized by airways obstruction and reduced maximum expiratory flow from the lungs that manifests as persistent daily symptoms, such as shortness of breath (dyspnoea), and limitation of the ability to perform daily activities or exertion. Furthermore, there are periodic exacerbations of the condition that result in worsening of the day-to-day symptoms and activity limitation, and can also lead to hospitalization of the patient because of the severity of the worsening symptoms/limitation. In addition, there is a progressive decline in lung function (disease progression) over several years.

Bronchodilator treatment in COPD includes but is not necessarily limited to reducing symptoms, particularly dyspnoea, to allow a patient to undertake more daily activities and other activities that require exertion, and preventing exacerbations.

Asthma is a chronic condition, which is characterized by widespread, variable and reversible airflow obstruction. Symptoms include coughing, wheezing, breathlessness and/or a tight feeling in the chest. Asthma attacks are generally caused by exposure to a trigger, such as pollen, dust or other allergens, which causes constriction of the airways (bronchoconstriction). It will be appreciated that a subject suffering from a condition such as asthma, may variously from time to time display no overt symptoms of the condition, or may suffer from periodic attacks during which symptoms are displayed or may experience exacerbations or worsening of the condition. In this context the term ‘treatment’ is intended to encompass prevention of such periodic attacks or exacerbations of the existing condition. Such treatment may be referred to as ‘maintenance treatment’ or ‘maintenance therapy’.

In one embodiment, the dry powder inhalers of the present invention are useful for the treatment of asthma or COPD.

The present invention provides dry powder inhalers comprising the compound of formula (I) and optionally the compound of formula (II), or a pharmaceutically acceptable salt thereof. Administration may be via the mouth or nose. In one embodiment, inhalation is via the mouth.

In one embodiment, the compound of formula (I) and specifically (4-[hydroxy(diphenyl)methyl]-1-{2-[(phenylmethyl)oxy]ethyl}-1-azoniabicyclo[2.2.2]octane bromide, may be administered by dry powder inhaler at a dose of about 31 to 32 mcg or about 15 to 16 mcg of the free cation, for example about 31.25 mcg or about 15.6 mcg of the free cation, and particularly 31.25 mcg and 15.625 mcg of the free cation, once-daily or twice-daily. In general, administration will be once-daily.

The compound of formula (II), or a pharmaceutically acceptable salt thereof, may for example be administered by inhalation at a dose of from about 1 mcg to about 400 mcg/day (calculated as the free base). In one embodiment, the compound of formula (II), or a pharmaceutically acceptable salt thereof, and specifically 4-{(1R)-2-[(6-{2-[(2,6-dichlorobenzyl)oxy]ethoxy}hexyl)amino]-1-hydroxyethyl}-2-(hydroxymethyl)phenol triphenylacetate, may be administered by dry powder inhaler at a dose of from about 1 mcg to 100 mcg/day, for example 3, 6.25, 12.5, 25, 50 or 100 mcg/day (calculated as the free base). In general, the compound of formula (II), or a pharmaceutically acceptable salt thereof, will be administered once-daily. In one embodiment, the compound of formula (II), or a pharmaceutically acceptable salt thereof, may be administered by dry powder inhaler at a dose of 12.5 mcg/day. In another embodiment, the compound of formula (II), or a pharmaceutically acceptable salt thereof, may be administered by dry powder at a dose of 25 mcg/day. In another embodiment, the compound of formula (II), or a pharmaceutically acceptable salt thereof, may be administered by dry powder inhaler at a dose of 50 mcg/day.

In a further embodiment, 4-{(1R)-2-[(6-{2-[(2,6-dichlorobenzyl)oxy]ethoxy}hexyl)amino]-1-hydroxyethyl}-2-(hydroxymethyl)phenol triphenylacetate, may be administered by dry powder inhaler, once-daily, at a dose of 25 mcg per day.

In a further embodiment, the present invention provides a dry powder inhaler comprising 4-{(1R)-2-[(6-{2-[(2,6-dichlorobenzyl)oxy]ethoxy}hexyl)amino]-1-hydroxyethyl}-2-(hydroxymethyl)phenol triphenylacetate present in an amount of about 25 mcg/dose, and (4-[hydroxy(diphenyl)methyl]-1-{2-[(phenylmethyl)oxy]ethyl}-1-azoniabicyclo[2.2.2]octane bromide present in an amount to deliver about 31.25 mcg/dose of the free cation.

In a further embodiment, the present invention provides a dry powder inhaler comprising 4-{(1R)-2-[(6-{2-[(2,6-dichlorobenzyl)oxy]ethoxy}hexyl)amino]-1-hydroxyethyl}-2-(hydroxymethyl)phenol triphenylacetate present in an amount of 25 mcg/dose, and (4-[hydroxy(diphenyl)methyl]-1-{2-[(phenylmethyl)oxy]ethyl}-1-azoniabicyclo[2.2.2]octane bromide present in an amount to deliver 31.25 mcg/dose of the free cation.

In a further embodiment, the present invention provides a dry powder inhaler comprising 4-{(1R)-2-[(6-{2-[(2,6-dichlorobenzyl)oxy]ethoxy}hexyl)amino]-1-hydroxyethyl}-2-(hydroxymethyl)phenol triphenylacetate present in an amount of about 25mcg/dose, and (4-[hydroxy(diphenyl)methyl]-1-{2-[(phenylmethyl)oxy]ethyl}-1-azoniabicyclo[2.2.2]octane bromide present in an amount to deliver about 15.625 mcg/dose of the free cation.

In a further embodiment, the present invention provides a dry powder inhaler comprising 4-{(1R)-2-[(6-{2-[(2,6-dichlorobenzyl)oxy]ethoxy}hexyl)amino]-1-hydroxyethyl}-2-(hydroxymethyl)phenol triphenylacetate present in an amount of 25 mcg/dose, and (4-[hydroxy(diphenyl)methyl]-1-{2-[(phenylmethyl)oxy]ethyl}-1-azoniabicyclo[2.2.2]octane bromide present in an amount to deliver 15.625 mcg/dose of the free cation.

The dry powder inhalers of the present invention may further comprise an inhaled corticosteroid, e.g. fluticasone propionate, mometasone furoate, budesonide or 6α,9α-difluoro-17α-[(2-furanylcarbonyl)oxy]-11β-hydroxy-16α-methyl-3-oxo-androsta-1,4-diene-17α-carbothioic acid S-fluoromethyl ester (fluticasone furoate).

In one embodiment, a dry powder inhaler of the present invention comprises 4-[hydroxy(diphenyl)methyl]-1-{2-[(phenylmethyl)oxy]ethyl}-1-azoniabicyclo[2. 2. 2]octane bromide present in an amount to deliver 31.25 mcg/dose or 15.625 mcg/dose of the free cation, 4-{(1R)-2-[(6-{2-[(2,6-dichlorobenzyl)oxy]ethoxy}hexyl)amino]-1-hydroxyethyl}-2-(hydroxymethyl)phenol triphenylacetate and 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate).

When the combination additionally includes an inhaled corticosteroid, this may be used at doses compatible with those known for monotherapy. When the inhaled corticosteroid is fluticasone furoate this may be administered by inhalation at a dose of from about 25 mcg to about 800 mcg daily, and if necessary in divided doses. Thus, the daily dose of fluticasone furoate may be for example 25, 50, 100, 200, 300, 400, 600 or 800 mcg, in general as a once-daily dose. In one embodiment, the daily dose of fluticasone furoate is 200 mcg. In a further embodiment, the daily dose of fluticasone furoate is 100 mcg. In yet a further embodiment, the daily dose of fluticasone furoate is 50 mcg.

The individual compounds of a dry powder inhaler as described herein may be administered either sequentially or simultaneously. When administered simultaneously the individual compounds may be in separate compositions or a combined composition (i.e. admixed). In general, such compositions will include pharmaceutical carriers or excipients as described hereinafter, but combinations of the compounds without any excipients are also within the ambit of this invention.

In a further embodiment, the present invention provides a dry powder inhaler comprising the compound of formula (I) and the compound of formula (II), or a pharmaceutically acceptable salt thereof, presented separately for sequential or simultaneous administration.

In yet a further embodiment, the present invention provides a dry powder inhaler comprising the compound of formula (I) and the compound of formula (II), or a pharmaceutically acceptable salt thereof, presented in admixture with each other for simultaneous administration.

In each of the two cases directed above, each of the compound of formula (I) and the compound of formula (II), or a pharmaceutically acceptable salt thereof, may be formulated with or without pharmaceutically acceptable carriers or excipients.

The present invention further provides a dry powder inhaler comprising the compound of formula (I) and the compound of formula (II), or a pharmaceutically acceptable salt thereof, wherein at least one of these compounds is formulated with a pharmaceutically acceptable carrier or excipient. The carrier(s) or excipient(s) for each compound may be the same or different.

The present invention further provides a dry powder inhaler comprising the compound of formula (I) and the compound of formula (II), or a pharmaceutically acceptable salt thereof, wherein each compound is formulated with a pharmaceutically acceptable carrier or excipient. The carrier(s) or excipient(s) for each compound may be the same or different.

When the dry powder inhaler of the invention additionally includes an inhaled corticosteroid, eg 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) this may likewise be formulated separately, either with or without one or more pharmaceutical carriers or excipients, and presented for either sequential or simultaneous administration, or the inhaled corticosteroid may be admixed with either the compound of formula (I) and/or the compound of formula (II). 6α,9α-Difluoro-17α-[(2-furanylcarbonyl)oxy]-11β-hydroxy-16α-methyl-3-oxo-androsta-1,4-diene-17β-carbothioic acid S-fluoromethyl ester may be formulated for example as described in WO02/12265, or as described hereinafter.

The compositions for delivery by a dry powder inhaler may be prepared by any of the methods well known in the art of pharmacy. In general, said methods include the step of bringing the active ingredient(s) into association with the carrier which constitutes one or more accessory ingredients. In general the compositions are prepared by uniformly and intimately bringing into association the active ingredient with liquid carriers or finely divided solid carriers or both and then, if necessary, shaping the product into the desired composition.

Active ingredients for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g. by micronization. The desired fraction may be separated out by air classification or sieving. Preferably, the particles will be crystalline.

Dry powder compositions generally contain a powder mix for inhalation of the active ingredient and a suitable powder base (carrier/diluent/excipient substance) such as mono-, di or poly-saccharides (e.g. lactose or starch). Use of lactose is preferred. The lactose may be for example anhydrous lactose or α-lactose monohydrate. In one embodiment, the carrier is α-lactose monohydrate.

Dry powder compositions according to the invention may comprise a carrier. The carrier when it is lactose e.g. α-lactose monohydrate, may form from about 91 to about 99%, e.g. 97.7-99.0% or 91.0-99.2% by weight of the formulation. In general, the particle size of the carrier, for example lactose, will be much greater than the inhaled medicament within the present invention. When the carrier is lactose it will typically be present as milled lactose, having a MMD (mass median diameter) of 60-90 μm.

The lactose component may comprise a fine lactose fraction. The ‘fine’ lactose fraction is defined as the fraction of lactose having a particle size of less than 7 μm, such as less than 6 μm, for example less than 5 μm. The particle size of the ‘fine’ lactose fraction may be less than 4.5 μm. The fine lactose fraction, if present, may comprise 2 to 10% by weight of the total lactose component, such as 3 to 6% by weight fine lactose, for example 4.5% by weight fine lactose.

Dry powder compositions may also include, in addition to the active ingredient and carrier, a further excipient (eg a ternary agent) such as a sugar ester, calcium stearate or magnesium stearate. Alternatively, the active ingredient may be presented without excipients.

Magnesium stearate, if present in the composition, is generally used in an amount of about 0.2 to 2%, e.g. 0.6 to 2% or 0.5 to 1.75%, e.g. 0.6%, 0.75%, 1%, 1.25% or 1.5% w/w, based on the total weight of the composition. The magnesium stearate will typically have a particle size in the range 1 to 50 μm, and more particularly 1-20 μm, e.g.1-10 μm. Commercial sources of magnesium stearate include Peter Greven, Covidien/Mallinckodt and FACI.

The present invention further provides a dry powder inhaler comprising the compound of formula (I) and the compound of formula (II), or a pharmaceutically acceptable salt thereof, wherein at least one of these two compounds is formulated with a pharmaceutically acceptable carrier and a ternary agent.

In another embodiment the present invention further provides a dry powder inhaler comprising the compound of formula (I) and the compound of formula (II), or a pharmaceutically acceptable salt thereof, wherein both compounds are formulated separately or together with a pharmaceutically acceptable carrier and a ternary agent.

The present invention further provides a dry powder inhaler comprising 4-{(1R)-2-[(6-{2-[(2,6-dichlorobenzyl)oxy]ethoxy]hexyl)amino]-1-hydroxyethyl}-2-(hydroxymethyl)phenol triphenylacetate and (4-[hydroxy(diphenyl)methyl]-1-{2-[(phenylmethyl)oxy ]ethyl}-1-azoniabicyclo[2.2.2]octane bromide each formulated separately with a pharmaceutically acceptable carrier and a ternary agent for sequential or simultaneous administration, wherein (4-[hydroxy(diphenyl)methyl]-1-{2-[(phenylmethyl)oxy]ethyl}-1-azoniabicyclo[2.2.2]octane bromide is present in an amount to deliver about 31.25 mcg/dose or 15.625 mcg/dose of the free cation.

In one embodiment said ternary agent is magnesium stearate.

The present invention further provides a dry powder inhaler comprising 4-{(1R)-2-[(6-{2-[(2,6-dichlorobenzyl)oxy]ethoxy}hexyl)amino]-1-hydroxyethyl}-2-(hydroxymethyl)phenol triphenylacetate and (4-[hydroxy(diphenyl)methyl]-1-{2-[(phenylmethyl)oxy]ethyl}-1-azoniabicyclo[2.2.2]octane bromide each formulated separately with lactose, as a pharmaceutically acceptable carrier, and magnesium stearate, as a ternary agent for sequential or simultaneous administration, wherein (4-[hydroxy(diphenyl)methyl]-1-{2-[(phenylmethyl)oxy]ethyl}-1-azoniabicyclo[2.2.2]octane bromide is present in an amount to deliver 31.25 mcg/dose or 15.625 mcg/dose of the free cation.

The dry powder inhalers of the present invention may be, for example, reservoir dry powder inhalers, unit-dose dry powder inhalers, or pre-metered multi-dose dry powder inhalers.

The compositions may be presented in unit dosage form for delivery by an appropriate dry powder inhaler. Dry powder compositions for topical delivery to the lung by inhalation may, for example, be presented in capsules and cartridges of for example gelatine, or blisters of for example laminated aluminium foil.

Each capsule, cartridge or blister may generally contain about 31 to 32 mcg or 15 to 16 mcg, for example about 31.25 mcg or about 15.6 mcg, such as 31.25 mcg or 15.625 mcg of the free cation of the compound of formula (I) and/or between 1 mcg-400 mcg, for example 1 to 100 mcg, such as 25 mcg of the compound of formula (II), or a pharmaceutically acceptable salt thereof. Packaging of the formulation may be suitable for unit dose or multi-dose delivery. As indicated above, the compound of formula (I) and the compound of formula (II), or a pharmaceutically acceptable salt thereof, may be formulated independently or in admixture. Said compounds may thus be incorporated in separate unit doses or may be combined in a single unit dose with or without additional excipients as deemed necessary.

In a further embodiment, each capsule, cartridge or blister may contain 31.25 mcg or 15.625 mcg of the free cation of the compound of formula (I) and/or 25 mcg of the compound of formula (II), or a pharmaceutically acceptable salt thereof.

In yet a further embodiment, each capsule, cartridge or blister may contain 31.25 mcg or 15.625 mcg of the free cation of (4-[hydroxy(diphenyl)methyl]-1-{2-[(phenylmethyl)oxy]ethyl}-1-azoniabicyclo[2.2.2]octane bromide and/or 25 mcg of 4-{(1R)-2-[(6-{2-[(2,6-dichlorobenzyl)oxy]ethoxy}hexyl)amino]-1-hydroxyethyl}-2-(hydroxymethyl)phenol triphenylacetate.

In one embodiment, a composition suitable for inhaled administration may be incorporated into a plurality of sealed dose containers provided on medicament pack(s) mounted inside an appropriate dry powder inhaler. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. Representative inhalation devices are the DISKHALER™ and DISKUS™ devices, marketed by GlaxoSmithKline. The DISKUS™ inhalation device is, for example, described in GB 2242134A.

A composition suitable for inhaled administration, may also be provided as a bulk reservoir in an inhalation device, the device then being provided with a metering mechanism for metering a dose of the composition from the reservoir to an inhalation channel where the metered dose is able to be inhaled by a patient inhaling at a mouthpiece of the device. Exemplary marketed devices of this type are TURBUHALER™ of AstraZeneca, TWISTHALER™ of Schering and CLICKHALER™ of Innovata.

A further delivery method for a dry powder inhalable composition is for metered doses of the composition to be provided in capsules (one dose per capsule) which are then loaded into an appropriate dry powder inhaler, typically by the patient on demand. The device has means to rupture, pierce or otherwise open the capsule so that the dose is able to be entrained into the patient's lung when they inhale at the device mouthpiece. As marketed examples of such devices there may be mentioned ROTAHALER™ of GlaxoSmithKline and HANDIHALER™ of Boehringer Ingelheim.

A dry powder composition may also be presented in a dry powder inhaler which permits separate containment of the compound of formula (I) and the compound of formula (II), or a pharmaceutically acceptable salt thereof, optionally in admixture with one or more excipients. Thus, for example, the individual compounds of the combination are administrable simultaneously but are stored separately, e.g. in separate pharmaceutical compositions, for example as described in WO 2003/061743 A1, WO 2007/012871 A1 and/or WO2007/068896. In one embodiment a delivery device permitting separate containment of actives is an inhaler device having two medicament packs in peelable blister strip form, each pack containing pre-metered doses in blister pockets arranged along its length. Said device has an internal indexing mechanism which, each time the device is actuated, peels opens a pocket of each strip and positions the packs so that each newly exposed dose of each pack is adjacent a manifold which communicates with a mouthpiece of the device. When the patient inhales at the mouthpiece, each dose is simultaneously drawn out of its associated pocket into the manifold and entrained via the mouthpiece into the patient's respiratory tract. Thus, each time the device is used, the patient is administered a combination therapy consisting of a dose from each medicament pack. A further device that permits separate containment of different compounds is DUOHALER™ of Innovata.

4-{(1R)-2-[(6-{2-[(2,6-dichlorobenzyl)oxy]ethoxy}hexyl)amino]-1-hydroxyethyl}-2-(hydroxymethyl)phenol, and its pharmaceutically acceptable salts, including 4-{(1R)-2-[(6-{2-[(2,6-dichlorobenzyl)oxy]ethoxy}hexyl)amino]-1-hydroxyethyl}-2-(hydroxymethyl)phenol triphenylacetate may be prepared as described in WO03/024439 (Example 78(i)), which is incorporated by reference herein.

4-[hydroxy(diphenyl)methyl]-1-{2-[(phenylmethyl)oxy]ethyl}-1-azoniabicyclo[2.2.2]octane bromide is described as Example 84 in WO2005/104745 (Glaxo Group Limited), which is incorporated by reference herein.

6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) is described as Example 1 in WO02/12265 (Glaxo Group Limited), which is incorporated by reference herein.

Clinical Studies

### 4-[hydroxy(diphenyl)methyl]-1-{2-[(phenylmethyl)oxy]ethyl}-1-azoniabicyclo[2.2.2]octane bromide

4-[hydroxy(diphenyl)methyl]-1-{2-[(phenylmethyl)oxy]ethyl}-1-azoniabicyclo[2.2.2]octane bromide has been found to be an effective long-acting potent, pan-active anti-muscarinic bronchodilator which demonstrates slow reversibility at the human M3 receptor in vitro and long duration of action in vivo when administered directly to the lungs in pre-clinical models. The long duration of action of this compound identified using in vitro models, when administered via inhalation in animals, and subsequently in early phase studies in healthy volunteers and COPD subjects supports the potential for use of this compound as a once daily bronchodilator for COPD.

Several clinical pharmacology studies have been conducted using 4-[hydroxy(diphenyl)methyl]-1-{2-[(phenylmethyl)oxy]ethyl}-1-azoniabicyclo[2.2.2]octane bromide in both healthy volunteers and COPD patients to investigate the safety, tolerability, pharmacokinetics and pharmacodynamics of this compound. The bronchodilatory effects and duration of action of single inhaled doses of this compound as measured by plethysmography (sG_{aw}, R_{aw}) and spirometry (FEV₁) were assessed in some of the above noted studies. These studies showed clinically relevant bronchodilation and 24 h duration of action for the compound.

Throughout, when the dose of 4-[hydroxy(diphenyl)methyl]-1-{2-[(phenylmethyl)oxy]ethyl}-1-azoniabicyclo[2.2.2]octane bromide is given, this relates to the active moiety 4-[hydroxy(diphenyl)methyl]-1-{2-[(phenylmethyl)oxy]ethyl}-1-azoniabicyclo[2.2.2]octane i.e the free cation rather than the salt.

In one such study, designed to evaluate the safety, efficacy and pharmacokinetics of 4-[hydroxy(diphenyl)methyl]-1-{2-[(phenylmethyl)oxy]ethyl}-1-azoniabicyclo[2.2.2]octane bromide in subjects with COPD, five once-daily doses (62.5 mcg, 125 mcg, 250 mcg, 500 mcg and 1000 mcg), taken over a 14-day treatment period, produced statistically significant improvements in pulmonary function compared to placebo. All once-daily doses showed numerically greater improvement in trough FEV₁ than the open label tiotropium active control (18 mcg once-daily). In addition, this study confirmed that 4-[hydroxy(diphenyl)methyl]-1-{2-[(phenylmethyl)oxy]ethyl}-1-azoniabicyclo[2.2.2]octane bromide has a once-daily profile.

A further study evaluated the efficacy and safety of three doses (125 mcg, 250 mcg and 500 mcg) of 4-[hydroxy(diphenyl)methyl]-1-{2-[(phenylmethyl)oxy]ethyl}-1-azoniabicyclo[2.2.2]octane bromide administered once-daily via a dry powder inhaler over a 28 day period in subjects with COPD. This study confirmed that 4-[hydroxy(diphenyl)methyl]-1-{2-[(phenylmethyl)oxy]ethyl}-1-azoniabicyclo[2.2.2]octane bromide appears to be safe and efficacious, maintaining significant bronchodilation over twenty four hours.

A further study shall evaluate the safety and efficacy of four doses (125 mcg, 62.5 mcg, 31.25 mcg and 15.6 mcg) administered once daily and two doses (31.25 mcg and 15.625 mcg) administered twice daily. Administration shall be via a dry powder inhaler.

### The Compound of Formula (II) (as the α-Phenylcinnamate Salt or the Triphenylacetate Salt)

The compound of formula (II) as the α-phenylcinnamate salt and the triphenylacetate salt has been studied in a number of clinical pharmacology studies, including single- and repeat-dose studies. In addition, these studies have evaluated the compound of formula (II) formulated with lactose and either cellobiose octaacetate or magnesium stearate.

In asthmatic patients, a statistically and clinically significant improvement in trough (24-hour) FEV1 was observed for all doses of the compound of formula (II) tested, compared to placebo. Single doses of 25 mcg to 100 mcg of the compound of formula (II) triphenylacetate (containing lactose and magnesium stearate) demonstrated 24 hour duration of action as assessed by a 200 mL or greater increase in mean 23 to 24 hour post-dose FEV1 versus placebo.

In COPD patients, treatment with 100 mcg and 400 mcg the compound of formula (II) alpha-phenylcinnamate (with lactose alone) achieved a clinically relevant adjusted mean difference from placebo in weighted mean trough FEV₁ (22 to 24 hrs) of >100 mL. Single doses of 25 mcg to 100 mcg of the compound of formula (II) triphenylacetate (containing lactose and magnesium stearate) demonstrated 24 hour duration of action as assessed by a 190 mL or greater increase in mean 23 to 24 hour post-dose FEV1 versus placebo).

### 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate)

Several clinical pharmacology studies have been conducted using 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) to investigate the safety and efficacy of this compound in asthmatic patients.

In one such study, the safety and efficacy of four doses of 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) in subjects with persistent uncontrolled asthma were evaluated. In this study, which was a randomised, double-blind, placebo-controlled, parallel group study, 598 patients received one of six treatments: fluticasone furoate (25, 50, 100 or 200 mcg) once daily, fluticasone propionate 100 mcg twice daily or placebo for 8 weeks. The primary endpoint was change from baseline in trough (pre-dose) forced expiratory volume in 1 second (FEV₁) at Week 8. At Week 8, relative to placebo fluticasone furoate 50-200 mcg once daily had significantly greater increases in trough FEV₁ from baseline (p<0.05) with fluticasone furoate 100 mcg and 200 mcg achieving a >200 mL increase. This study supports the use of fluticasone furoate (100 or 200 mcg once-daily) for the treatment of persistant uncontrolled asthma.

Combination Therapy

A combination of 4-[hydroxy(diphenyl)methyl]-1-{2-[(phenylmethyl)oxy]ethyl}-1-azoniabicyclo[2.2.2]octane bromide and the compound of formula (II) as the triphenylacetate salt has been administered to sixteen healthy Japanese volunteers, aged 20 to 65, as part of a clinical trial to assess the safety, tolerability, pharmacokinetics and pharmacodynamics of single inhaled doses of 4-[hydroxy(diphenyl)methyl]-1-{2-[(phenylmethyl)oxy]ethyl}-1-azoniabicyclo[2.2.2]octane bromide and the compound of formula (II) triphenylacetate as monotherapies and in combination. This study was a randomised, double blind, placebo-controlled, four-way crossover study wherein subjects received a single dose of:
4-[hydroxy(diphenyl)methyl]-1-{2-[(phenylmethyl)oxy]ethyl}-1-azoniabicyclo[2.2.2]octane bromide (500 mcg dose),
the compound of formula (II) triphenylacetate (50 mcg dose),
4-[hydroxy(diphenyl)methyl]-1-{2-[(phenylmethyl)oxy]ethyl}-1-azoniabicyclo[2.2.2]octane bromide (500 mcg dose) and the compound of formula (II) triphenylacetate (50 mcg dose) concurrently, or
placebo

at each of the four treatment periods. On enrollment into the study subjects were assigned to one of four treatment sequences based on a Williams design.

This clinical study in healthy Japanese volunteers, evaluated the effect of 4-[hydroxy(diphenyl)methyl]-1-{2-[(phenylmethyl)oxy]ethyl}-1-azoniabicyclo[2.2.2]octane bromide (500 mcg dose) and the compound of formula (II) triphenylacetate (50 mcg dose) administered as single inhaled doses and concurrently (4-[hydroxy(diphenyl)methyl]-1-{2-[(phenylmethyl)oxy]ethyl}-1-azoniabicyclo[2.2.2]octane bromide (500 mcg dose) and the compound of formula (II) triphenylacetate (50 mcg dose)) on lung function parameters. Single inhaled doses and the combination administered using dry powder inhalers were found to be well tolerated. In this study FEV₁ values were recorded. FEV₁ values were higher for all treatment groups compared with placebo. The group dosed with 4-[hydroxy(diphenyl)methyl]-1-{2-[(phenylmethyl)oxy]ethyl}-1-azoniabicyclo[2.2.2]octane bromide (500 mcg dose) and the compound of formula (II) triphenylacetate (50 mcg dose) concurrently showing the largest difference relative to placebo.

Pharmaceutical Formulations

Preparation of Blends

### 4-[hydroxy(diphenyl)methyl]-1-{2-[phenylmethyl)oxy]ethyl}-1-azoniabicyclo[2.2.2]octane bromide

Throughout, when the dose of 4-[hydroxy(diphenyl)methyl]-1-{2-[(phenylmethyl)oxy]ethyl}-1-azoniabicyclo[2.2.2]octane bromide is given, this relates to the active moiety 4-[hydroxy(diphenyl)methyl]-1-{2-[(phenylmethyl)oxy]ethyl}-1-azoniabicyclo[2.2.2]octane i.e the free cation rather than the salt.

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns). The level of fines in the α-lactose monohydrate, which can be measured by laser diffraction may be 4.5% less than 4.5 micron.

4-[hydroxy(diphenyl)methyl]-1-{2-[(phenylmethyl)oxy]ethyl}-1-azoniabicyclo[2.2.2]octane bromide is micronised before use in an APTM microniser to give a mass median diameter of 1 to 5 microns, such as 2 to 5 microns.

Pharmaceutical grade magnesium stearate, sourced from Peter Greven, complying with the requirements of Ph.Eur/USNF may be used as supplied with a mass median particle size of 8 to 12 microns.

Blend A

Lactose monohydrate may be passed through a sieve and then combined with magnesium stearate and blended using either a high shear mixer (a QMM, PMA or TRV series mixer, such as TRV25 or TRV65) or a low shear tumbling blender (a Turbula mixer) to provide a magnesium stearate/lactose premix, hereinafter referred to as blend A.

Blend B

Final blend B may be obtained as follows. An quantity of blend A and 4-[hydroxy(diphenyl)methyl]-1-{2-[(phenylmethyl)oxy]ethyl}-1-azoniabicyclo[2.2.2]octane bromide may be screened, for example using a COMIL™, and then blended with the remaining blend A using either a high shear mixer (a QMM, PMA or TRV series mixer, such as TRV25 or TRV65) or a low shear tumbling blender (a Turbula mixer).

### Representative Batch Formula for 4-[hydroxy(diphenyl)methyl]-1-{2-[(phenylmethyl)oxy]ethyl}-1-azoniabicyclo[2.2.2]octane bromide Powder Blend (62.5 microgram per blister)

| Ingredient | Quantity | |
|---|---|---|
| Micronised 4-[hydroxy(diphenyl)methyl]-1- | 74.1 | g |
| {2-[(phenylmethyl)oxy]ethyl}-1- | | |
| azoniabicyclo[2.2.2]octane bromide | | |
| Magnesium Stearate | 75 | g |
| Lactose Monohydrate | To 12.5 | kg |

Note:
74.1 g is equivalent to 62.5 g of the free cation. The quantity of 4-[hydroxy(diphenyl)methyl]-1-{2-[(phenylmethyl)oxy]ethyl}-1-azoniabicyclo[2.2.2]octane bromide added may be adjusted to reflect the assigned purity of the input drug substance.

Powder blends of 4-[hydroxy(diphenyl)methyl]-1-{2-[(phenylmethyl)oxy]ethyl}-1-azoniabicyclo[2.2.2]octane bromide for blisters containing other quantities of active, such as 31.25 mcg or 15.625 mcg per blister, may be prepared using the same procedure.

Blending Parameters (Using a TRV25, 12.5 kg Scale)

| Blend | Time (mins) | Approximate Speed (rpm) |
|---|---|---|
| A | 6 | 460 |
| B | 10 | 590 |

Blister Strip Preparation

The blended composition may then be transferred into blister strips (typical nominal mean quantity of blend per blister is 12.5-13.5 mg) of the type generally used for the supply of dry powder for inhalation and the blister strips were sealed in the customary fashion.

The Compound of Formula (II) Triphenylacetate

Pharmaceutical grade α-lactose monohydrate, which can be sourced from DMV Fronterra Excipients, complying with the requirements of Ph.Eur/USNF may be used. Before use, the α-lactose monohydrate may be sieved through a coarse screen (typical mesh size 500 microns). The level of fines in the α-lactose monohydrate, which can be measured by laser diffraction may be 4.5% less than 4.5 micron.

The compound of formula (II) triphenylacetate is micronised before use in an APTM microniser to give a MMD (mass median diameter) of from 1 to 5 microns, such as 2 to 5 microns, for example 1.8 microns.

Pharmaceutical grade Magnesium stearate, which can be sourced from Peter Greven, complying with the requirements of Ph.Eur/USNF may be used as supplied with a mass median particle size 8 to 12 microns.

Blend A

Lactose monohydrate may be passed through a sieve and then combined with magnesium stearate (typically 130 g) and blended using either a high shear mixer (a QMM, PMA or TRV series mixer, such as TRV25 or TRV65) or a low shear tumbling blender (a Turbula mixer) to provide a magnesium stearate/lactose premix, hereinafter referred to as blend A.

Blend B

Final blend B may be obtained as follows. An appropriate quantity of blend A and the compound of formula (II) triphenylacetate (typically 5-165 g) may be screened, for example using a COMIL™, and then blended with the remaining blend A using either a high shear mixer (a QMM, PMA or TRV series mixer) or a low shear tumbling blender (a Turbula mixer). The final concentration of the compound of formula (II) triphenylacetate in the blends is typically in the range 0.02% w/w 0.8% w/w free base equivalent.

Blister Strip Preparation

The blended composition is transferred into blister strips (typical nominal mean quantity of blend B per blister is 12.5-13.5 mg) or the type generally used for the supply of dry powder for inhalation and the blister strips are then sealed in the customary fashion.

### EXAMPLE PREPARATIONS

Using the above-described procedure the following exemplary formulations may be prepared:

| | | Mass of the | | |
|---|---|---|---|---|
| | Mass of | compound of formula | | |
| Blend | Magnesium | (II) triphenylacetate | Mass of | Quantity per |
| No | stearate | (micronised) | lactose | blister |
| 1 | 130 g | 5.0 g | To 13 kg | 13 mg |
| 2 | 130 g | 10.3 g | To 13 kg | 13 mg |
| 3 | 130 g | 20.7 g | To 13 kg | 13 mg |
| 4 | 130 g | 41.3 g | To 13 kg | 13 mg |
| 5 | 130 g | 82.7 g | To 13 kg | 13 mg |
| 6 | 130 g | 165.4 g | To 13 kg | 13 mg |

Note:
The quantity of the compound of formula (II) triphenylacetate used is based on a base to salt conversion factor of 1.59. For example, 41 g of the compound of formula (II) triphenylacetate is equivalent to 25 g of the free base.

### EXAMPLE BLENDING PARAMETERS (USING A TRV25, 13 KG SCALE, THE COMPOUND IF FORMULA (II) TRIPHENYLACETATE POWDER BLEND (25 MICROGRAM BLISTER))

| Blend | Time (mins) | Approximate Speed (rpm) |
|---|---|---|
| A | 9 | 550 |
| B | 8.5 | 550 |

### 6α,9α-difluoro-17α-11β-furanylcarbonyl)oxy]-11βhydroxy-16α-methyl-3-oxo-androsta-1,4-diene-17β-carbothioic acid S-fluoromethyl ester (fluticasone furoate)

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns). The level of fines in the α-lactose monohydrate, which can be measured by Laser Diffraction, may be 5 to 8% less than 4.5 micron.

### 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate)_is micronised before use in an APTM microniser to give a mass median diameter of 1 to 5 microns.

Blend

Lactose monohydrate may be passed through a sieve and then blended with 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) using either a high shear mixer (a QMM, PMA or TRV series mixer, such as TRV25 or TRV65) or a low shear tumbling blender (a Turbula mixer).

### Representative Batch Formula for 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate)

Powder Blend (100 Microgram Per Blister)

| | |
|---|---|
| Ingredient | Quantity |

| | | |
|---|---|---|
| 6α,9α-difluoro-17α-[(2-furanylcarbonyl)oxy]- | 84.0 | g |
| 11β-hydroxy-16α-methyl-3-oxo-androsta-1,4- | | |
| diene-17β-carbothioic acid S-fluoromethyl ester | | |
| (fluticasone furoate) | | |
| Lactose Monohydrate | To 10.5 | kg |

### Representative Batch Formula for 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate)

Powder Blend (200 Microgram Per Blister)

| | |
|---|---|
| Ingredient | Quantity |

| | | |
|---|---|---|
| 6α,9α-difluoro-17α-[(2-furanylcarbonyl)oxy]- | 168.0 | g |
| 11β-hydroxy-16α-methyl-3-oxo-androsta-1,4- | | |
| diene-17β-carbothioic acid S-fluoromethyl ester | | |
| (fluticasone furoate) | | |
| Lactose Monohydrate | To 10.5 | kg |

Blending Parameters (Using a TRV25, 10.5 kg Scale)

| | Time (mins) | Approximate Speed (rpm) |
|---|---|---|
| Blend | 7 | 550 |

Blister Strip Preparation

The blended composition may then be transferred into blister strips (typical nominal mean quantity of blend per blister is 12.5-13.5 mg) of the type generally used for the supply of dry powder for inhalation and the blister strips were sealed in the customary fashion.

Powder blends of 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) _for blisters containing other quantities of active, such as 25 mcg or 50 mcg per blister, may be prepared using the same procedure.

### EXAMPLE DRY POWDER INHALER DEVICES

4-[hydroxy(diphenyl)methyl]-1-{2-[(phenylmethyl)oxy]ethyl}-1-azoniabicyclo[2.2.2]octane bromide may be administered by a dry powder inhaler containing one or two blister strips. One or two strips each contain a blend of micronised 4-[hydroxy(diphenyl)methyl]-1-{2-[(phenylmethyl)oxy]ethyl}-1-azoniabicyclo[2.2.2]octane bromide (31.25 or 15.625 mcg per blister), magnesium stearate and lactose monohydrate.

4-[hydroxy(diphenyl)methyl]-1-{2-[(phenylmethyl)oxy]ethyl}-1-azoniabicyclo[2.2.2]octane bromide may be administered by a dry powder inhaler containing one or two blister strips. One or two strips each contain a blend of micronised 4-[hydroxy(diphenyl)methyl]-1-{2-[(phenylmethyl)oxy]ethyl}-1-azoniabicyclo[2.2.2]octane bromide (31.25 or 15.625 mcg per blister), magnesium stearate (at an amount of 0.6% w/w of the total powder weight per blister) and lactose monohydrate. The DPI device will deliver, when actuated, the contents of a single blister simultaneously from each of the two blister strips. Each blister strip is a double foil laminate containing 30 blisters per strip.

4-[hydroxy(diphenyl)methyl]-1-{2-[(phenylmethyl)oxy]ethyl}-1-azoniabicyclo[2.2.2]octane bromide and the compound of formula (II) triphenylacetate may be administered by a dry powder inhaler containing two blister strips. One strip contains a blend of micronised 4-[hydroxy(diphenyl)methyl]-1-{2-[(phenylmethyl)oxy]ethyl}-1-azoniabicyclo[2.2.2]octane bromide (31.25 or 15.625 mcg per blister), magnesium stearate and lactose monohydrate. The second strip contains a blend of micronised 4-{(1R)-2-[(6-{2-[(2,6-dichlorobenzyl)oxy]ethoxy}hexyl)amino]-1-hydroxyethyl}-2-(hydroxymethyl)phenol triphenylacetate (25 mcg per blister), magnesium stearate and lactose monohydrate.

In a further embodiment, 4-[hydroxy(diphenyl)methyl]-1-{2-[(phenylmethyl)oxy]ethyl}-1-azoniabicyclo[2.2.2]octane bromide and 4-{(1R)-2-[(6-{2-[(2,6-dichlorobenzyl)oxy]ethoxy}hexyl)amino]-1-hydroxyethyl}-2-(hydroxymethyl)phenol triphenylacetate may be administered by a dry powder inhaler containing two blister strips, wherein one strip contains a blend of micronised 4-[hydroxy(diphenyl)methyl]-1-{2-[(phenylmethyl)oxy]ethyl}-1-azoniabicyclo[2.2.2]octane bromide (31.25 or 15.625 mcg per blister), magnesium stearate (at an amount of 0.6% w/w of the total powder weight per blister) and lactose monohydrate. The second strip contains a blend of micronised 4-{(1R)-2-[(6-{2-[(2,6-dichlorobenzyl)oxy]ethoxy}hexyl)amino]-1-hydroxyethyl}-2-(hydroxymethyl)phenol triphenylacetate (25 mcg per blister), magnesium stearate (at an amount of 1.0% w/w of the total powder weight per blister) and lactose monohydrate. The DPI device will deliver, when actuated, the contents of a single blister simultaneously from each of the two blister strips. Each blister strip is a double foil laminate containing 30 blisters per strip.

In a further embodiment, 4-[hydroxy(diphenyl)methyl]-1-{2-[(phenylmethyl)oxy]ethyl}-1-azoniabicyclo[2.2.2]octane bromide and 4-{(1R)-2-[(6-{2-[(2,6-dichlorobenzyl)oxy]ethoxy}hexyl)amino]-1-hydroxyethyl}-2-(hydroxymethyl)phenol triphenylacetate may be administered by a dry powder inhaler containing two blister strips, wherein one strip contains a blend of micronised 4-[hydroxy(diphenyl)methyl]-1-{2-[(phenylmethyl)oxy]ethyl}-1-azoniabicyclo[2.2.2]octane bromide (31.25 or 15.625 mcg per blister), 4-{(1R)-2-[(6-{2-[(2,6-dichlorobenzyl)oxy]ethoxy}hexyl)amino]-1-hydroxyethyl}-2-(hydroxymethyl)phenol triphenylacetate (25 mcg per blister), magnesium stearate and lactose monohydrate. The second strip contains a blend of 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate) at an amount of 100 or 200 mcg per blister, and lactose monohydrate. The DPI device will deliver, when actuated, the contents of a single blister simultaneously from each of the two blister strips. Each blister strip is a double foil laminate containing 7, 14 or 30 filled blisters per strip.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A dry powder inhaler comprising the compound of formula (I): 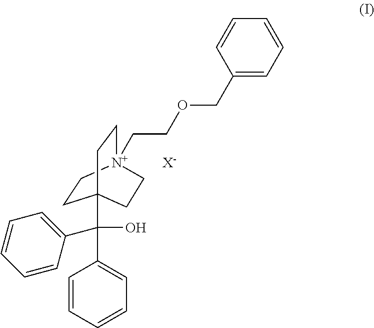
wherein X⁻ is a pharmaceutically acceptable anion selected from the group consisting of chloride, bromide, iodide, hydroxide, sulfate, nitrate, phosphate, acetate, trifluoroacetate, fumarate, citrate, tartrate, oxalate, succinate, mandelate, methanesulfonate and p-toluenesulfonate, and wherein the free cation of the compound of formula (I) is present in an amount of about 31 to 32 mcg/dose or about 15 to 16 mcg/dose.

2-4. (canceled)

5. A dry powder inhaler according to claim 1, which further comprises a compound of formula (II): 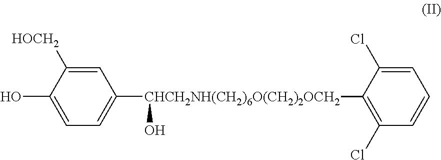
or a pharmaceutically acceptable salt thereof.

6. (canceled)

7. A dry powder inhaler according to claim 1, wherein the compound of formula (I) is
4-[hydroxy(diphenyl)methyl]-1-{2-[(phenylmethyl)oxy]ethyl}-1-azoniabicyclo[2.2.2]octane bromide present in an amount of 31.25 mcg/dose or 15.625 mcg/dose.

8. (canceled)

9. A dry powder inhaler according to claim 5 wherein the compound of formula (II) is 4-{(1R)-2-[(6-{2-[(2,6-dichlorobenzyl)oxy]ethoxy}hexyl)amino]-1-hydroxyethyl}-2-(hydroxymethyl)phenol triphenylacetate, present in an amount of 25 mcg/dose.

10. A dry powder inhaler according to claim 1 selected from the group consisting of: a reservoir dry powder inhaler, a unit-dose dry powder inhaler, and a pre-metered multi-dose dry powder inhaler.

11. A dry powder inhaler according to claim 1 wherein the compound of formula (I) and the compound of formula (II), or a pharmaceutically acceptable salt thereof, are presented as separate or admixed compositions.

12-13. (canceled)

14. A dry powder inhaler according to claim 11 wherein each composition of the compound of formula (I) and the compound of formula (II), or a pharmaceutically acceptable salt thereof, contains a carrier, wherein the carrier is lactose.

15-16. (canceled)

17. A dry powder inhaler according to claim 11 wherein each composition contains a ternary agent, wherein the

18-19. (canceled)

20. A dry powder inhaler as claimed in claim 17, wherein magnesium stearate is present in an amount of about 0.6% w/w in a composition of the compound of formula (I), and/or an amount of about 1.0% w/w in a composition of the compound of formula (II), or a pharmaceutically acceptable salt thereof.

21-24. (canceled)

25. A dry powder inhaler according to claim 1 further comprising an inhaled corticosteroid.

26. A dry powder inhaler according to claim 25 wherein the inhaled corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), present in an amount of about 100 mcg/dose or about 200 mcg/dose.

27-35. (canceled)
